(19) United States
(12) Patent Application Publication
(10) Pub. No.: US 2010/0221300 A1
(43) Pub. Date: Sep. 2, 2010

(54) Processing of Angiogenic Scaffolds for Large Organ Regeneration

(76) Inventors: Brendan Harley, Cambridge, MA (US); Harry K. Reddy, Lexington, MA (US); Ioannis V. Yannas, Newton, MA (US); Christopher Zagorski, Newton, MA (US)

Correspondence Address: Pearl Cohen Zedek Latzer, LLP, 1500 Broadway, 12th Floor, New York, NY, 10036, US

(21) Appl. No.: 12/084,253

(22) PCT Filed: Oct. 16, 2006
(86) PCT No.: PCT/US06/40235

§ 371 Date: Jun. 8, 2009

Related U.S. Application Data
(60) Provisional application No. 60/730,880, filed on Oct. 28, 2005.

(51) Int. Cl. A61K 9/00; A61K 35/12; A61K 38/18; B29C 35/02; B29C 35/08; A61P 17/02

(52) U.S. Cl. 424/422; 424/93.1; 514/12; 264/28; 264/494; 424/93.7

(57) ABSTRACT

This invention relates to a method for fabricating large scaffolds in a variety of shapes with an organized pore structure. The pore structure is organized such that pores are generally aligned perpendicular to the edges of the scaffold, regardless of-the particular macroscopic scaffold shape. Specifically, a freeze-drying based fabrication method for creating large, polymeric porous scaffolds for tissue engineering applications, with an organized pore structure of columnar pores extending from the scaffold periphery into the main mass of the scaffold.

### FIELD OF INVENTION

This invention relates to a method for fabricating large scaffolds in a variety of shapes with an organized pore structure. The pore structure is organized such that pores are generally aligned perpendicular to the edges of the scaffold, regardless of the particular macroscopic scaffold shape.

### BACKGROUND OF THE INVENTION

Implanting a scaffold to regenerate lost or damaged tissue, requires the use of a scaffold that supports adequate cell migration into and around the scaffold, short-term support of these cells following implantation with an adequate supply of oxygen and nutrients and long-term angiogenesis and remodeling of the scaffold (degradation of the scaffold and remodeling of the vasculature and tissue architecture). These functions should be supported for new stroma and tissue formation.

Scaffolds are prefabricated supports, which may be seeded with cells. While cells can easily adsorb into the outermost portions of the scaffold, cell distributions may not be uniform throughout the scaffold due to random motility and limitations in the diffusion of nutrients. This in turn may lead to uneven and distorted regeneration of tissue, which, if allowed to persist, may create other pathologies. Even if cells are homogenously distributed throughout a large-scale scaffold, there is a need for a vascular supply to nourish the cells in the interior of the scaffold, since these cells are positioned in a location within the scaffold, which is not readily accessible to the surrounding vasculature and are therefore deprived of nutrients and oxygen necessary for their long term viability. Cell survival necessitates it being within the diffusion distance of a capillary, for the formation of a concentration gradient facilitating an exchange whereby the cell can receive an adequate concentration of oxygen and nutrients. While a vascular supply can grow into an implanted scaffold from surrounding vascularized tissue, the angiogenic process takes time, which may result in cell death in the scaffolding interior, prior to adequate vascularization.

One of the limitations to date in successful tissue engineering is a lack of an appropriate material and architecture whereby complex tissues may be assembled, in particular providing the ability of appropriate cells to align themselves along desired characteristic dimension to form a functioning tissue. Thus scaffolds, which can provide a size/scale large enough to be applicable for use in the regeneration of breast or of other organs are lacking.

While, collagen-based scaffolds fabricated via lyophilization (where a suspension of collagen and an acid, is frozen in a pan-shaped, or tubular mold and sublimated to produce sheets (1-3 mm thick) of porous scaffold), the macroscopic shapes obtained by these methods (i.e., small tubes or thin sheets) are familiar. To date, no method producing porous scaffolds with significantly larger characteristic lengths or scaffolds with organized pore structures that influence cell migration as well as nutrient transport into the scaffold have been introduced.

### SUMMARY OF THE INVENTION

In one embodiment, the invention provides a solid, porous scaffold for implantation, comprising an organic polymer, having a width of at least 3.5 mm in at least one direction, and pores oriented perpendicular to an edge of said scaffold. In one embodiment, the scaffold comprises pores situated closer to a surface of said scaffold having a diameter, which is greater than pores situated further from said surface

In one embodiment, this invention provides a process for preparing a solid, porous, biocompatible scaffold having a width of at least 3.5 mm in at least one direction, and pores oriented perpendicular to an edge of said scaffold, the process comprising the steps of:
a) applying a polymeric mixture to a mold comprised of a conductive material, wherein said mold has at least 2 components
b) immersing the suspension-filled mold in (a) in a super-cooled refrigerant held at a constant temperature, for a period of time until said suspension is solidified, whereby ice crystals are formed in said solidified suspension, said crystals being oriented perpendicular to an edge of said scaffold;
c) exposing a portion of said solidified suspension to conditions which enable sublimation in said portion, whereby pores are formed which are perpendicular to an edge of said scaffold; and
d) removing the remaining components of said mold to expose said solid porous scaffold.

In another embodiment, this invention provides a scaffold produced according to the processes of the invention.

In one embodiment, the invention provide a method of organ or tissue engineering in a subject, comprising the step of implanting a scaffold of the inevntion, including in one embodiment, in application to wound healing.

In one embodiment, the invention provides a method of organ or tissue repair or regeneration in a subject, comprising the step of implanting a scaffold. of the invention.

### DETAILED DESCRIPTION OF THE INVENTION

The invention is directed to solid gradient scaffolds, methods of producing the same, and therapeutic applications arising from their utilization.

Scaffolds are in one embodiment, porous materials used for a variety of tissue engineering applications; one major application of porous scaffolds is as templates that induce regeneration of lost or damaged tissue. In order to treat larger tissues and organs (characteristic length scale>1 mm), it is necessary to develop technologies able to produce scaffolds with significantly larger characteristic lengths, an organized pore structure, and in a variety of macroscopic shapes to suit each implant site.

In one embodiment, the term “porous” refers to a matrix that comprises holes or voids, rendering the material permeable In another embodiment, the scaffold is non-uniformly porous. In one embodiment, non-uniformly porous scaffolds allow for permeability at some regions, and not others, within the scaffold, or in another embodiment, the extent of permeability differs within the scaffold.

In one embodiment, this invention provides a solid, porous scaffold for implantation, comprising an organic polymer, having a width of at least 3.5 mm in at least one direction, and pores oriented perpendicular to an edge of the scaffold.

In one embodiment, the term “scaffold” or “scaffolds” refers to three-dimensional structures that assist in the tissue regeneration process by providing a site for cells to attach, proliferate, differentiate and secrete an extra-cellular matrix, eventually leading to tissue formation. In one embodiment, a scaffold provides a support for the repair, regeneration or generation of a tissue or organ.

In one embodiment, the scaffold comprises a biocompatible material, which, in another embodiment may comprise carbohydrate, or in another embodiment, proteins or specific amino acids, or in another embodiment, a biocompatible polymer or monomer as described herein, or in another embodiment, a combination thereof.

In one embodiment, the scaffold comprises at least one polymer, which is a natural polymer, or in another embodiment an organic polymer, or in another embodiment, an extracellular matrix protein, or in another embodiment an analogue thereof, or in another embodiment, a combination thereof.

In one embodiment, the polymer is a copolymer. In another embodiment, the polymer is a homo- or, in another embodiment heteropolymer. In another embodiment, the polymer of this invention are natural polymer. In another embodiment, the polymer is a free radical random copolymer, or, in another embodiment, a graft copolymer. In one embodiment, the polymer may comprise proteins, peptides or nucleic acids.

In one embodiment, a graft copolymer of two different extracellular matrix components is formed, such as for example a type I collagen and GAG. The final ratio of collagen/GAG may be equal, in another embodiment, to any combination between 85/15 to 100/0 w/w by methods well known in the art (Yannas, et al. PNAS 1989, 86:933)). According to this aspect of the invention, and in one embodiment, a length of the polymer is then exposed to a concentration gradient of a collagenase, for a period of time, wherein time, in another embodiment, is varied, which may, in another embodiment, provide for greater digestion of for example collagen, in some sections of the scaffold thus exposed. In one embodiment, digestion is a function of enzyme concentration, or in another embodiment, exposure time to a given concentration, or in another embodiment, a combination thereof.

For example, and in one embodiment, the scaffold is comprised of a graft copolymer of a type I collagen and a GAG, whose ratio is controlled by adjusting the mass of the macromolecules mixed to form the copolymer.

In another embodiment, the polymer may comprise a biopolymer such as, for example, collagen. In another embodiment, the polymer may comprise a biocompatible polymer such as polyesters of [alpha]-hydroxycarboxylic acids, such as poly(L-lactide) (PLLA) and polyglycolide (PGA); polymer disclosed in U.S. Pat. No. 6,333,029 or 6,355,699; or any bioresorbable and biocompatible polymer, co-polymer or mixture of polymer or co-polymer known in the art, or hereinafter discovered, which perform or function substantially similarly.

In one embodiment, the biodegradable polymer comprise functional groups such as esters, anhydrides, orthoesters, and amides. In another embodiment, the polymer biodegrades rapidly such as for example in one embodiment poly[lactide-co-glycolide], polyanhydrides, and polyorthoesters. In one embodiment, bioerodible polymers include polylactides, polyglycolides, and copolymers thereof, poly(ethylene terephthalate), poly(butyric acid), poly(valeric acid), poly(lactide-co-caprolactone), poly(lactide-co-glycolide), polyanhydrides, polyphosphazenes, poly(.epsilon.-caprolactone), poly(dioxanone), poly(hydroxybutyrate), poly(hydroxyvalerate), polyorthoesters, blends, and copolymers thereof. Examples of biodegradable and biocompatible polymers of acrylic and methacrylic acids or esters include poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), etc. Other polymers which can be used in the present invention include polyalkylenes such as polyethylene and polypropylene; polyarylalkylenes such as polystyrene; poly(alkylene glycols) such as poly(ethylene glycol); poly(alkylene oxides) such as poly(ethylene oxide); and poly(alkylene terephthalates) such as poly(ethylene terephthalate). Additionally, polyvinyl polymers can be used which include polyvinyl alcohols, polyvinyl ethers, polyvinyl esters, and polyvinyl halides. Exemplary polyvinyl polymers include poly(vinyl acetate), polyvinyl phenol, and polyvinylpyrrolidone. It is to be understood that any combination of polymers as described herein may be used in the scaffolds and methods of this invention and represent an embodiment thereof.

In another embodiment, the polymers comprise in one embodiment extracellular matrix (ECM) component. In another embodiment, the ECM component is purified from tissue, by means well known in the art. For example, if collagen is desired, in one embodiment, the naturally occurring extracellular matrix can be treated to remove substantially all materials other than collagen. The purification may be carried out to substantially remove, or in another embodiment, enrich for glycoproteins, or in another embodiment glycosaminoglycans, or in another embodiment proteoglycans, or in another embodiment lipids, or in another embodiment non-collagenous proteins or in another embodiment nucleic acid (DNA or RNA), by methods known to one skilled in the art.

In one embodiment, the polymer may comprise Type I collagen, Type II collagen, Type IV collagen, gelatin, agarose, cell-contracted collagen containing proteoglycans, glycosaminoglycans or glycoproteins, fibronectin, laminin, elastin, fibrin, synthetic polymeric fibers made of poly-acids such as polylactic, polyglycolic or polyamino acids, polycaprolactones, polyamino acids, polypeptide gel, copolymers thereof and/or combinations thereof.

In one embodiment, the polymers may comprise a functional group, which enables linkage formation with other molecules of interest, some examples of which are provided further hereinbelow. In one embodiment, the functional group is one, which is suitable for hydrogen bonding (e.g., hydroxyl groups, amino groups, ether linkages, carboxylic acids and esters, and the like).

In another embodiment, functional groups may comprise an organic acid group. In one embodiment, the term “organic acid group” is meant to include any groupings which contain an organic acidic ionizable hydrogen, such as carboxylic and sulfonic acid groups. The expression “organic acid functional groups” is meant to include in one embodiment, any group that function in a similar manner to organic acid groups under specific reaction conditions, for instance metal salts of such acid groups, such as, for example alkali metal salts like lithium, sodium and potassium salts, or alkaline earth metal salts like calcium or magnesium salts, or quaternary amine salts of such acid groups, such as, for example quaternary ammonium salts.

In one embodiment, functional groups may comprise acid-hydrolyzable bonds including ortho-ester or amide groups. In another embodiment, functional groups may comprise base-hydrolyzable bonds including alpha-ester or anhydride groups. In another embodiment, functional groups may comprise both acid- or base-hydrolyzable bonds including carbonate, ester, or iminocarbonate groups. In another embodiment, functional groups may comprise labile bonds, which are known in the art and can be readily employed in the methods/processes and scaffolds described herein (see, e.g. Peterson et al., Biochem. Biophys. Res. Comm. 200(3): 1586-159 (1994) 1 and Freel et al., J. Med. Chem. 43: 4319-4327 (2000)).

In another embodiment, the scaffold further comprises a pH-modifying compound. In one embodiment, the term “pH-modifying” refers to an ability of the compound to change the pH of an aqueous environment when the compound is placed in or dissolved in that environment. The pH-modifying compound, in another embodiment, is capable of accelerating the hydrolysis of the hydrolyzable bonds in the polymer upon exposure of the polymer to moisture and/or heat. In one embodiment, the pH-modifying compound is substantially water-insoluble. Suitable substantially water-insoluble pH-modifying compounds may include substantially water-insoluble acids and bases. Inorganic and organic acids or bases may be used, in other embodiments.

In one embodiment, as described herein, other molecules may be incorporated within the scaffold, which may, in another embodiment, be attached via a functional group, as herein described. In another embodiment, the molecule is conjugated directly to the scaffold.

In one embodiment, the extracellular matrix proteins comprise a collagen, a glycosaminoglycan, or a combination thereof. In another embodiment, the polymers of this invention may comprise extracellular matrix components, such as hyaluronic acid and/or its salts, such as sodium hyaluronate; glycosaminoglycans such as dermatan sulfate, heparan sulfate, chondroiton sulfate and/or keratan sulfate; mucinous glycoproteins (e.g., lubricin), vitronectin, tribonectins, surface-active phospholipids, rooster comb hyaluronate. In some embodiments, the extracellular matrix components may be obtained from commercial sources, such as ARTHREASE™ high molecular weight sodium hyaluronate; SYNVISC® Hylan G-F 20; HYLAGAN® sodium hyaluronate; HEALON® sodium hyaluronate and SIGMA® chondroitin 6-sulfate.

In another embodiment, one or more biomolecules may be incorporated in the scaffold. The biomolecules may comprise, in other embodiments, drugs, hormones, antibiotics, antimicrobial substances, dyes, radioactive substances, fluorescent substances, silicone elastomers, acetal, polyurethanes, radiopaque filaments or substances, anti-bacterial substances, chemicals or agents, including any combinations thereof. The substances may be used to enhance treatment effects, reduce the potential for implantable article erosion or rejection by the body, enhance visualization, indicate proper orientation, resist infection, promote healing, increase softness or any other desirable effect.

In one embodiment, the scaffold varies in terms of its polymer concentration, or concentration of and component of the scaffold, including biomolecules and/or cells incorporated within the scaffold.

In one embodiment, the biomolecule may comprise chemotactic agents; antibiotics, steroidal or non-steroidal analgesics, anti-inflammatories, immunosuppressants, anti-cancer drugs, various proteins (e.g., short chain peptides, bone morphogenic proteins, glycoprotein and lipoprotein); cell attachment mediators; biologically active ligands; integrin binding sequence; ligands; various growth and/or differentiation agents (e.g., epidermal growth factor, IGF-I, IGF-II, TGF-β I-III, growth and differentiation factors, vascular endothelial growth factors, fibroblast growth factors, platelet derived growth factors, insulin derived growth factor and transforming growth factors, parathyroid hormone, parathyroid hormone related peptide, bFGF; TGFβ superfamily factors; BMP-2; BMP-4; BMP-6; BMP-12; sonic hedgehog; GDF5; GDF6; GDF8; PDGF); small molecules that affect the upregulation of specific growth factors; tenascin-C; hyaluronic acid; chondroitin sulfate; fibronectin; decorin; thromboelastin; thrombin-derived peptides; heparin-binding domains; heparin; heparan sulfate; DNA fragments, DNA plasmids, or any combination thereof.

In one embodiment growth factors include heparin binding growth factor (hbgf), transforming growth factor alpha or beta (TGF.beta.), alpha fibroblastic growth factor (FGF), epidermal growth factor (TGF), vascular endothelium growth factor (VEGF), some of which are also angiogenic factors. In another embodiment factors include hormones such as insulin, glucagon, and estrogen. In some embodiments it may be desirable to incorporate factors such as nerve growth factor (NGF) or muscle morphogenic factor (MMF). In one embodiment, TNF alpha/beta, or Matrix metalloproteinases (MMP's) are incorporated.

In another embodiment, the scaffold may comprise one or more of an autograft, an allograft and a xenograft of any tissue with respect to the subject. In one embodiment, the tissue is a homogenate, which in one embodiment comprises the scaffold used to repair, or in one embodiment, regenerate the same tissue, such as in one embodiment to grow bone tissue.

In another embodiment, the scaffold implanted may further comprise cells. In one embodiment, the cells are seeded on said scaffold, or in another embodiment, on the periphery of the scaffold. In another embodiment, the cells are stem or progenitor cells. In another embodiment, the method further comprises the step of administering cytokines, growth factors, hormones or a combination thereof to the subject.

In one embodiment, the scaffolds may comprise cells. In one embodiment, the cells may include one or more of the following: chondrocytes; fibrochondrocytes; osteocytes; osteoblasts; osteoclasts; synoviocytes; bone marrow cells; mesenchymal cells; stromal cells; stem cells; embryonic stem cells; precursor cells derived from adipose tissue; peripheral blood progenitor cells; stem cells isolated from adult tissue; genetically transformed cells; a combination of chondrocytes and other cells; a combination of osteocytes and other cells; a combination of synoviocytes and other cells; a combination of bone marrow cells and other cells; a combination of mesenchymal cells and other cells; a combination of stromal cells and other cells; a combination of stem cells and other cells; a combination of embryonic stem cells and other cells; a combination of progenitor cells isolated from adult tissue and other cells; a combination of peripheral blood progenitor cells and other cells; a combination of stem cells isolated from adult tissue and other cells; and a combination of genetically transformed cells and other cells. In another embodiment, any of these cells for use in the scaffolds and methods of the invention, may be engineered to express a desired molecule, such as for example heparin binding growth factor (hbgf), transforming growth factor alpha or beta (TGF.beta.), alpha fibroblastic growth factor (FGF), epidermal growth factor (TGF), vascular endothelium growth factor (VEGF), some of which are also angiogenic factors. In another embodiment expressed factors include hormones such as insulin, glucagon, and estrogen. In another embodiment factors such as nerve growth factor (NGF) or muscle morphogenic factor (MMF), or in another embodiment, TNF alpha/beta are expressed.

In one embodiment, the scaffolds of this invention is porous, wherein said pores vary in one embodiment in terms of depth, which may range in another embodiment, from about 1 to about 35000 μm, and in one embodiment, in terms of diameter, which may range in another embodiment from about 0.75 to about 1500 μm, or in another embodiment, a combination thereof, within said scaffold. In one embodiment, the ratio between the length of the scaffold and the height of the scaffold is larger than 10.

In one embodiment, the term “about” refers to a deviation from the range of 1-20%, or in another embodiment, of 1-10%, or in another embodiment of 1-5%, or in another embodiment, of 5-10%, or in another embodiment, of 10-20%.

In one embodiment the pores vary in diameter from about 1 to about 100 μm, or in another embodiment, from 100 to about 200 μm, or in another embodiment, from 200 to about 300 μm, or in another embodiment, from 300 to about 400 μm, or in another embodiment, from 400 to about 500 μm, or in another embodiment, from 500 to about 750 μm, or in another embodiment, from 750 to about 1000 μm, or in another embodiment, from 1000 to about 1500 μm, or in another embodiment, from 1500 to about 2000 μm, or in another embodiment, from 2000 to about 2500 μm, or in another embodiment, from 2500 to about 3000 μm, or in another embodiment, from 3000 to about 3500 μm

In one embodiment the pores vary in diameter from about 1 to about 100 μm, or in another embodiment, from 100 to about 200 μm, or in another embodiment, from 200 to about 300 μm, or in another embodiment, from 300 to about 400 μm, or in another embodiment, from 400 to about 500 μm, or in another embodiment, from 500 to about 750 μm, or in another embodiment, from 750 to about 1000 μm.

In another embodiment, the invention provides a solid porous scaffold, in which the pores form a channel, where, in another embodiment, the channels are distributed on the face of the scaffold, or in another embodiment, oriented along an axis, which in another embodiment, the width of said channel is greater at a point more proximal to the scaffold surface, than to its core. In one embodiment, the channel narrows from the periphery of the scaffold to its center. In some embodiments, pores situated closer to a surface of said scaffold have a diameter which is greater than pores situated further from said surface.

In one embodiment the pores, or in another embodiment channels formed by the pores are designed for a particular tissue formation, such as in one embodiment for regeneration of intestine tissue, or in another embodiment for kidney, or in another embodiment for bone, or in another embodiment for breast, or in another embodiment innervated tissue.

In one embodiment, the width of the channel varies between about 1 μm to 5 cm, or in another embodiment between about 1 μm to 200 μm, or in another embodiment between about 1 μm to 200 μm, or in another embodiment between about 200 μm to 400 μm, or in another embodiment between about 400 μm to 600 μm, or in another embodiment between about 600 μm to 800 μm, or in another embodiment between about 800 μm to 1 mm, or in another embodiment between about 1 mm to 5 mm, or in another embodiment, between 5 mm to about 1 cm, or in another embodiment, between 1 cm and 2 cm, or in another embodiment, between 2 cm and 3 cm, or in another embodiment, between 3 cm and 4 cm, or in another embodiment, between 4 cm and 5 cm.

In one embodiment, scaffolds that are non-uniformly porous are especially suited for tissue engineering, repair or regeneration, wherein the tissue is a connector tissue, or wherein the scaffold is utilized to engineer, repair or regenerate two or three, or more, tissues in close proximity to one another. A difference in porosity may facilitate migration of different cell types to the appropriate regions of the scaffold, in one embodiment. In another embodiment, a difference in porosity may facilitate development of appropriate cell-to-cell connections among the cell types comprising the scaffold, required for appropriate structuring of the developing/repairing/regenerating tissue. For example, dendrites or cell processes extension may be accommodated more appropriately via the varied porosity of the scaffolding material. In another embodiment, the permeability differences in the scaffolding material may prevent and enhance protein penetrance, wherein penetration is a function of molecular size, such that the lack of uniform porosity serves as a molecular sieve. It is to be understood that the gradient scaffolding of this invention may be used any purpose for which non-uniform porosity is desired, and is to be considered as part of this invention.

In another embodiment, the scaffold varies in its average pore diameter and/or distribution thereof. In another embodiment, the average diameter of the pores varies as a function of its spatial organization in said scaffold. In another embodiment, the average diameter of the pores varies as a function of the pore size distribution along an arbitrary axis of the scaffold. In another embodiment, the scaffold comprises regions devoid of pores. In another embodiment, the regions are impenetrable to molecules greater than 1000 Da in size.

In one embodiment, the term “average pore diameter” refers to area average diameter, D_{3,2}. In another embodiment, D_{3,2} is a measure of average pore diameter and in another embodiment follows a lognormal distribution. In one embodiment the term “D_{3,2}” refers to the average diameter of the pores calculated assuming spherical pores and inferring the average diameter from the surface area exposed to the measuring device.

In one embodiment, lognormal distribution will be determined according to the following formula for calculating the frequency distribution:

$$df = \frac{1}{σ \sqrt{2 π}} exp [ - \frac{{( {d}_{p} - {d}_{A} )}^{2}}{2 {σ}^{2}} ] {dd}_{p}$$

### Wherein:

dₚ is the pore diameter in μM
d_{A} is the average pore diameter
a is the standard deviation of pore sizes in μM

In one embodiment, the term “pore size distribution refers to σ, the standard deviation of pore sizes in μM.

In this aspect of the invention, and in one embodiment, the scaffolds of the invention vary in terms of their cross-link density. In another embodiment cross-link density may be modified by any crosslinking technology known in the art. In one embodiment the term “cross link density” refers to the average number of monomers between each cross-link. In another embodiment, the lower the number of monomers between cross links, the higher the cross link density, which, in another embodiment affects the physic-chemical properties of the scaffold. The cross-linking density should be controlled in one embodiment, so as to obtain a pore size large enough to allow cell migration. In another embodiment, pore size may be determined by scanning electron microscopy or in another embodiment, by using macromolecular probes. A cell suspension containing cells such as, in one embodiment, keratinocytes, chrondocytes and osteoblasts, is injected into the polymer network along with suitable growth factors. The cells would then be allowed to grow within the network. As the cells grow the network around them would degrade. The timing of the network degradation should coincide with the cells forming their own network (organ/tissue) through inter-cell contacts.

In one embodiment, the invention provides a process for preparing a solid scaffold, wherein the process further comprises exposing said scaffold to a cross-linking agent after applying a polymeric suspension to a mold comprised of a conductive material, wherein said mold has at least 2 components. In another embodiment, the cross-linking agent is glutaraldehyde, or in another embodiment formaldehyde, or in another embodiment paraformaldehyde, or in another embodiment formalin, (1 ethyl 3-(3-dimethyl aminopropyl)carbodiimide (EDAC), or in another embodiment UV light, or in another embodiment, a combination thereof. In one embodiment the exposure time vary to control the cross-link density as described hereinabove. In one embodiment, super-cooling the polymeric suspension under conditions inducing a gradient as described herein, creates a scaffold wherein the cross link density varies throughout the scaffold.

In one embodiment, the size and shape of said scaffold is a function of the tissue into which the scaffold is to be implanted.

In another embodiment, the scaffold, when implanted, promotes angiogenesis within, or proximal to the scaffold.

In one embodiment the scaffold is comprised of a material whose stiffness is sufficient to resist compressive forces of tissue proximal to a site of implantation. In another embodiment, the degree of cross-linking of the scaffold material is adjusted to compensate for the compressive forces of the surrounding tissue. In one embodiment, initial polymer slurry concentration is varied as a function of the compressive force of the target tissue. In one embodiment, the scaffold comprises plasticizers which impart some elasticity to the scaffold, yet preventing scaffold collapse. In one embodiment the scaffolds are so constructed so that the plasticizer is concentrated at the surface of the scaffold, or in another embodiment the concentration of the plasticizer will vary in depth and distribution to add elasticity and improve resistance to the compressive force of the surrounding target tissue.

In another embodiment, the plasticizer may be any substance of molecular weight lower than that of the biocompatible polymer that creates an increase in the free volume. In one embodiment, the plasticizer is an organic compound, which in one embodiment is triglyceride of varying chain length, or in another embodiment, the plasticizer is water.

In one embodiment, the scaffold is fabricated using a process that creates an amorphous glassy-state solid, comprised of a biocompatible polymer. In one embodiment “glassy-state solid” refers to an amorphous metastable solid wherein rapid removal of a plasticizer causes increase in viscosity of the biopolymer to the point where translational mobility of the critical polymer segment length is arrested and alignment corresponding to the polymer's inherent adiabatic expansion coefficient is discontinued.

In one embodiment, preparation of an amorphous glassy-state solid is accomplished by rapid cooling of an aerated melt of the biocompatible polymer, or in another embodiment by rapid solvent removal under vacuum, or in another embodiment, by freeze-drying. In one embodiment, preparing an amorphous glassy-state solid is accomplished by extrusion, which in one embodiment is at temperatures higher than 65° C. or, in another embodiment, at temperatures between about 4 and about 40° C. In one embodiment, width, length, depth, or a combination thereof, of the surface folds are designed into the dye used for extrusion, in conjunction with extrusion conditions. It will be understood by a skilled person in the art, that any process capable of producing amorphous glass with high portion of interconnected porosity (sponge-like) where the pore size is controllable by varying the fabrication conditions is appropriate for use for producing a scaffold of this invention and is thus within the scope of the invention.

In one embodiment, scaffolds are prepared according to the processes of this invention, in a highly porous form, by freeze-drying and sublimating the material. This can be accomplished by any number of means well known to one skilled in the art, such as, for example, that disclosed in U.S. Pat. No. 4,522,753 to Dagalakis, et al. For examples, porous gradient scaffolds may be accomplished by lyophilization. In one embodiment, extracellular matrix material may be suspended in a liquid. The suspension is then frozen and subsequently lyophilized. Freezing the suspension causes the formation of ice crystals from the liquid. These ice crystals are then sublimed under vacuum during the lyophilization process thereby leaving interstices in the material in the spaces previously occupied by the ice crystals. The material density and pore size of the ro resultant scaffold may be varied by controlling, in other embodiments, the rate of freezing of the suspension and/or the amount of water in which the extracellular matrix material is suspended at the initiation of the freezing process.

According to this aspect of the invention and in one embodiment, to produce scaffolds having a relatively large, uniform pore size and a relatively low material density, the extracellular matrix suspension may be frozen at a slow, controlled rate (e.g., −0.1° C./min or less) to a temperature of about −20° C., followed by lyophilization of the resultant mass. To produce scaffolds having a relatively small uniform pore size and a relatively high material density, the extracellular matrix material may be tightly compacted by centrifuging the material to remove a portion of the liquid (e.g., water) in a substantially uniform manner prior to freezing. Thereafter, the resultant mass of extracellular matrix material is flash-frozen using liquid nitrogen followed by lyophilization of the mass. To produce scaffolds having a moderate uniform pore size and a moderate material density, the extracellular matrix material is frozen at a relatively fast rate (e.g., >−1° C./min) to a temperature in the range of −20 to −40° C. followed by lyophilization of the mass.

In another embodiment, this invention provides a process for preparing a solid, porous, biocompatible scaffold having a width of at least 3.5 mm in at least one direction, and pores oriented perpendicular to an edge of said scaffold, the process comprising the steps of applying a polymeric suspension to a mold comprised of a conductive material, wherein said mold has at least 2 components; super-cooling the suspension-filled multicomponent mold in the previous step in a refrigerant held at a constant temperature, for a period of time until said suspension is solidified, whereby ice crystals are formed in said solidified suspension, said crystals being oriented perpendicular to an edge of said scaffold; exposing a portion of said solidified polymeric suspension by removing at least one component to conditions which enable sublimation in said portion, whereby pores are formed which are perpendicular to an edge of said scaffold; and removing the remaining components of said mold to expose said solid porous scaffold, thereby preparing a solid, porous, biocompatible scaffold.

In another embodiment, the invention provides a process for preparing any of the scaffolds described herein.

In one embodiment, “polymeric suspension” or “suspension” refers to any suspended system that would form a solid scaffold upon removal of one phase in the system. In one embodiment, the suspended system is a suspension, or in another embodiment an emulsion, or in another embodiment, a gel or in another embodiment, a foam. In one embodiment, the polymeric suspension is comprised of monomers or in another embodiment, single biocompatible molecules.

In one embodiment, the invention provides a process for preparing a solid scaffold, wherein the process further comprises super-cooling the suspension-filled mold in after applying a polymeric suspension to a mold, at a constant temperature, for a period of time until said suspension is solidified, whereby ice crystals are formed in said solidified suspension, said crystals being oriented perpendicular to an edge of said scaffold. In another embodiment, the solidified suspension or a portion thereof, is exposed to conditions which enable sublimation in the exposed region, where in another embodiment, pores are formed perpendicular to an edge of said scaffold, or in another embodiment, the pores formed vary in their diameter in the exposed region, relative to the unexposed region.

In one embodiment, the porous scaffold has “tunnels” which may be oriented in another embodiment from the periphery to the core of the scaffold, such that in one embodiment, the diameter of the tunnel narrows as a function of the distance from the periphery. In another embodiment the tunnels is the result of removal of mold components creating open tunnels leading from the periphery of the scaffold into the center of the scaffold. In one embodiment, removable of specific mold components, which in one embodiment may be at the surface of the scaffold, or, in another embodiment, at the interior and conditions for solidifying the suspension, may be such that tunnels are created.

According to this aspect of the invention, and in one embodiment, in order to produce a gradient scaffolding of this invention, the freezing rate is controlled, such that a thermal gradient is created within the scaffold, during its formation. For example, a slurry of interest comprising polymers as described and/or exemplified herein, may be inserted in a supercooled silicone oil bath, as described by Loree et al. (1989) Proc. 15^{th} Annual Northeast Bioeng. Conf., pp. 53-54). According to this aspect, in one embodiment, the container is only partially immersed, and is not completely submerged in the bath, such that a freezing front which travels up the length of the container is created, thereby creating a temperature gradient within the slurry.

In another embodiment, a solar bath effect is used to control ice crystallization rate and size in the mold, which facilitate control of the pore size in the lyophilized scaffold mass. In one embodiment, a solute is incorporated into the mass and a temperature gradient is induced by placing the pan containing the mass on a cold plate, which in one embodiment may be the freeze-dryer shelf, or in another embodiment a heat lamp may be placed on top of the pan. Since solubility is a function of temperature, a solute concentration gradient will result. In another embodiment, solute concentration affects the freezing temperature, resulting in different crystal size in a fixed freezing time, which, in a gradually concentrated solute will result in graduated porosity with pore size inversely proportional to the direction of increased solute concentration. In one embodiment, the solute comprises heterogeneous nucleation centers for water.

In one embodiment, the gradient is preserved by halting the freezing process prior to achieving thermodynamic equilibrium. The means for determining the time to achieving thermodynamic equilibrium in a slurry thus immersed, when in a container with a given geometry, will be readily understood by one skilled in the art. Upon achieving the desired temperature gradient, the slurry, in one embodiment, is removed from the bath and subjected to freeze-drying. Upon sublimation, the remaining material is the scaffolding comprising the polymer, with a gradient in its average pore diameter.

In another embodiment, a gradient in freezing rate of the scaffold is generated with the use of a graded thermal insulation layer between the container, which contains the scaffold components, and a shelf in a freezer on which the container is placed. In one embodiment, a gradient in the thermal insulation layer is constructed via any number of means, well known in the art, such as, for example, the construction of a thicker region in the layer along a particular direction, or in another embodiment, by varying thermal conductivity in the layer. The latter may be accomplished via use of, for example, aluminum and copper, or plexiglass and aluminum, and others, all of which represent embodiments of the present invention.

In one embodiment, the invention provides a process for preparing a solid porous, biocompatible scaffold of the invention which utilizes a mold with at least 2 components. In one embodiment, a multicomponent mold of a size and shape approximating the tissue into which said scaffold is to be implanted is used. In one embodiment, mold is comprised of two or more conductive materials, where, in another embodiment, the conductive materials differ in terms of their heat transfer coefficient, leading to difference in local rates of freezing during the super-cooling of the polymeric suspension. In one embodiment, each component of the multicomponent mold is comprised of a different conductive material.

In one embodiment, the invention provides a scaffold prepared according to the process described herein.

In another embodiment, the process further comprises the step of exposing the scaffold to a gradient of solutions, which are increased in their concentration of an enzyme, which degrades or solubilizes at least one extracellular matrix component. According to this aspect of the invention, and in one embodiment, digestion of at least one extracellular matrix component increases as a function of increasing enzyme concentration.

In one embodiment, the step of locally decreasing Tg to below that of the storage temperature, is followed by a change in environmental condition, increasing Tg such that the scaffold's Tg is above the storage temperature. In another embodiment, increasing Tg to above the storage conditions is achieved by dehydration of the matrix, which in one embodiment is done by exposing the matrix to temperatures lower than the desired Tg, or in another embodiment, by exposing the matrix to saturated salt solutions. In one embodiment, the saturated salt solution used is Lithium Chloride (LiCl), or in another embodiment Potassium Acetate (K⁺CH₃COO⁻) or in another embodiment to Phosphorous Pentoxide (P₂O₅), or in another embodiment to a concentration of Sulfuric acid imparting relative humidity values of below 0.35. In one embodiment, when exceeding Tg is achieved by locally heating the scaffold, removal of the heating element will result in local cooling of the scaffold material to below Tg, thereby inhibiting further pore collapse according to the methods of the invention. In one embodiment, increasing Tg may involve cross-linking of the scaffold material, thereby increasing the critical segment length (x).

In one embodiment, controlled pore collapse is conducted along an axis of the scaffold. In one embodiment, water evaporation from regions of interest may be accomplished at appropriate pressure known in the art, such as, for example, through the use of hot air directed at the region. According to this aspect of the invention, the dried regions will be devoid of pores, or in another embodiment, will be diminished in terms of the extent of porosity in the region, by the controlled collapse of these pores, due to surface tension issues.

In one embodiment, the term degrade/s or solubilizes encompasses partial degradation or solubilization, or in another embodiment, complete degradation or solubilization.

In one embodiment, the invention provides a method of organ or tissue engineering in a subject, comprising the step of implanting a scaffold of this invention.

In another embodiment, this invention provides a method of organ or tissue repair or regeneration in a subject, comprising the step of implanting a scaffold of this invention in a subject.

According to these aspects of the invention, and in one embodiment, the scaffold may be one produced by a process of this invention.

In one embodiment, this invention provides an implantable gradient scaffold, which may have varying mechanical properties to fit the application as to the desired implantation site of the scaffold. For instance, the pore size and the material density may be varied to produce a scaffold having a desired mechanical configuration. In particular, such variation of the pore size and the material density of the scaffold is particularly useful when designing a scaffold which provides for a desired amount of cellular migration therethrough, while also providing a desired amount of structural rigidity. In addition, according to the concepts of the present disclosure, implantable devices can be produced that not only have the appropriate physical microstructure to enable desired cellular activity upon implantation, but also has the biochemistry (collagens, growth factors, glycosaminoglycans, etc.) naturally found in tissues where the scaffolding is implanted for applications such as, for example, tissue repair or regeneration.

In one embodiment, the method of the invention is used for wound healing.

In one embodiment, the term “wound” refers to damaged biological tissue in the most general sense. In another embodiment, the wound is a laceration of the skin. In one embodiments, the wound may be an abrasion of the skin with two separated parts of tissue which in another embodiment, need to be brought together. In one embodiments, the wound may refer to a surgical incision. In another embodiment, the wound may involve damage to lung tissue, arterial walls, or other organs with elastic fibers. In one embodiment, the wound may involve an abscess, or in another embodiment, the wound may be exacerbated by diabetes. In one embodiment, the methods and scaffolds of the invention are used to accelerate wound healing.

According to this aspect of the invention and in one embodiment, wound healing may comprise fibrin clot formation, recruitment of inflammatory cells, reepitheliazation, and matrix formation and remodeling and as such, the scaffolds of this invention in one embodiment or the methods in another, may incorporate molecules involved in these stages with the scaffold. In another embodiment, immediately after tissue injury, blood vessel disruption leads to the extravasation of blood and concomitant platelet aggregation and blood coagulation resulting in fibrin clot formation and similarly, the scaffolds of this invention in one embodiment or the methods in another, may incorporate molecules involved in this stage, or in another embodiment, its facilitation. Activated platelets trapped within the fibrin clot degranulate and release a variety of cytokines and growth hormones. These cytokines and growth hormones help to recruit inflammatory cells to the site of injury, to stimulate angiogenesis, and to initiate the tissue movements associated with reepitheliazation and connective tissue contraction. In one embodiment, the scaffold of the invention is comprised of invaginated surface topography, allowing for regrowth of disrupted blood vessels. In another embodiment, the scaffold further comprises cytokines and growth hormones.

In one embodiment, neutrophils and monocytes are recruited to the site of injury by a number of chemotactic signals including in another embodiment the growth factors and cytokines released by the degranulating platelets, formyl methionyl peptides cleaved from bacterial proteins, and the by-products of proteolysis of fibrin and other matrix proteins. Neutrophil infiltration ceases in one embodiment after a few days, but macrophages continue to accumulate by continued recruitment of monocytes to the wound site. Activated macrophages release growth factors and cytokines thereby amplifying the earlier signals from the degranulating platelets.

In another embodiment, formation of granulation tissue and reepithelialization of the wound starts. Reepithelialization is performed in one embodiment, by the basal keratinocytes which lose their attachments to the basal lamina and crawl over the provisional matrix of fibrin and fibronectin, and underlying matrix, followed by epidermal cells reproduction—thereby providing the replacement cells needed. Keratinocyte proliferation is regulated by keratinocyte growth factor and members of the epidermal growth factor (EGF) family, which in another embodiment are incorporated into the scaffolds, or in one embodiment, the methods of the invention. In order to migrate through the fibrin clot, the keratinocytes must dissolve the fibrin barrier in front of them. Plasmin is the chief fibrinolytic enzyme used in this process and as such may be incorporated in one embodiment into the scaffold of the invention and used in the methods of the invention in another embodiment. Reepitheliazation is made easier by the underlying contractile connective tissue, which shrinks to bring the wound margins toward one another. Epidermal migration ceases when the wound surface has been covered by a monolayer of cells.

In one embodiment, cells of the new epidermis undergo the standard differentiation program of cells in the outer layers of unwounded epidermis. A new stratified epidermis is, thereby, reestablished from the margins of the wound inward. Matrix formation and remodeling begins simultaneously with reepithelialization. The matrix is constantly altered over the next several months with the elimination of the fibronectin from the matrix and the accumulation of collagen that provides the residual scar with increasing tensile strength. As such molecules involved in inhibition of excessive scaring, or in one embodiment enzymes facilitating elimination of fibronectin, such as in another embodiment MMP's, may be incorporated in one embodiment into the scaffold of the invention and used in the methods of the invention in another embodiment. Elastin fibers, which are responsible for the elasticity of tissue, are only detected in human scars years after the injury. In one embodiment, the gradient scaffold of the invention is seeded with epidermis cells at the periphery of the scaffold and implanted into the wound, thereby accelerating healing of the wound.

According to this aspect of the invention and in one embodiment, a solid, porous, biocompatible gradient scaffold, seeded with epidermal cells and further comprising one or more extracellular matrix components or analogs thereof, is used to heal an open wound by implanting the scaffold into the wound, wherein the scaffold comprises elastin, neutrophils, monocytes and EGF. In another embodiment, the scaffold is additionally seeded with stem cells, which in one embodiment are engineered to express growth factors.

In one embodiment, the method and solid gradient scaffold of the invention is used for regeneration of breast tissue, following breast augmentation procedure. In another embodiment, following the incision and insertion of the gradient scaffold of the invention, inflammatory exudate starts to flow into the large open pore channels at the scaffold surface within the first few hours following implantation. Fibrin, formed from fibrinogen condenses creating a network on which blood vessels can grow. Other factors and cells present in exudate help reconstruct the stroma within the scaffold and promote angiogenesis. The vasculature in the pre-existing tissue becomes closer to the scaffold due to contraction of the surrounding tissue and increased pressure from the space taken up by the implant. An additional vascular network is also formed surrounding the scaffold as capsule forms. The high concentration of angiogenic factors in exudate and from migrating/seeded cells causes blood vessels to grow into the scaffold, supporting the nearby cells indefinitely.

The following examples are presented in order to more fully illustrate the preferred embodiments of the invention. They should in no way be construed, however, as limiting the broad scope of the invention

### EXAMPLES

### Example 1

### Regeneration of a Large 3D Volume of Breast Tissue

The scaffold used is a sphere which is 50 mm in diameter, the pore structure form open channels at or near the surface which extend to the center of the scaffold. The diameter of these channels increases from the center to the scaffold surface, with the diameter near the surface as high as a few millimeters. As the channels extend toward the center they may divide to form a network of channels inside the scaffold, mimicking the progressive division of blood vessels in tissue. The scaffold is seeded with appropriate cells in the periphery. The cells extend from the outer surface to an approximate depth of 10 mm inside the scaffold. The scaffold also has VEGF bound onto the collagen fibers. The diameter of the pore channels at the scaffold surface is 1 mm.

The procedure for implanting the device is analogous to the procedure used to implant saline-filled breast implants. Saline-filled breast implants are fully coated implants available in fixed-volume, with a thick shell, a peripheral seam, and an internal septation, which divide the implant into compartments, intended to minimize bulging of one part of the implant when another part was compressed. Texturing of implant shells is intended to reduce capsular contracture. The incisions are made either directly below the nipple/areolar complex, in the crease below the breast, or in the axillary region, depending on the patient's anatomy and preference. The implants are usually placed underneath the pectorals (chest) muscle, as saline implants in this location give the breast a much more natural feel and appearance. Nearly all breast tissue may be visualized in mammograms with the implants under the muscle; less so when it is placed over the muscle

The scaffold is inserted by using a trans-axillary approach. The device is placed above the pectoralis major muscle. The placement of the scaffold above pectoralis major is expected to increase capsular contracture, and help bring the vascular bed in close proximity with the scaffold surface. The patient is placed under general anesthetic. Following the axillary incision the surgeon creates a small pocket to insert the scaffold between the breast gland tissue and pectoralis major. The scaffold is inserted into the space formed and the incision is closed. Following surgery the patient wears a specially designed undergarment to protect the device from being dislodged and from excessive compressive force. Pain medications are utilized as necessary following surgery. Once in place, the pressure from the surrounding tissue brings the existing vasculature in contact with the device's outer surface, forcing tissue into contact with the scaffold. The formation of capsule around the implant occurs spontaneously, creating multiple layers of fibrous tissue containing a variable amount of contractile cells, the innermost layers contain vasculature which is brought in close proximity to the scaffold. The degree to which capsule forms around the implant is dependant on the material from which it is composed, forming more around synthetic polymers.

Inflammatory exudate is released from capillaries in phases, bathing the wound created by the incision, in plasma proteins. Different cell types are recruited over time to remove damaged tissue, induce the formation of new tissue, reconstruct damaged matrix, basement membrane and connective tissue, and establish a new blood supply. Fluid exudate is released in three phases following injury: the first phase begins almost immediately after injury and involves a histamine-stimulated release of fluid and lasts anywhere between 8 to 30 minutes. The next phase is similar beginning straight after the first; it lasts longer, up to several days. The final phase commences a few hours after injury and the effects become maximal in 2-3 days, gradually resolving over a matter of weeks. Cellular exudate is produced in the second and third phases. The general make-up of the matrix becomes more fluid, allowing the contents of the exudate to diffuse more easily, but a sudden increase in tissue pressure doesn't occur. This will help exudate flow through the pores and channels in the scaffold, without a sudden increase in pressure damaging the implant. The components of exudate, both cellular and molecular (as detailed earlier) aid in angiogenesis and the regeneration of tissue.

The inflammatory exudate starts to flow into the large open pore channels at the scaffold surface within the first few hours following implantation. Fibrin, formed from fibrinogen condenses, creating a network on which blood vessels can grow. Other factors and cells present in exudate help reconstruct the stroma within the scaffold and promote angiogenesis. The vasculature in the pre-existing tissue comes closer to the scaffold due to contraction of the surrounding tissue and increased pressure from the space taken up by the implant. An additional vascular network is formed surrounding the scaffold as capsule forms. The high concentration of angiogenic factors in exudate and from migrating/seeded cells causes blood vessels to grow into the scaffold, supporting the nearby cells indefinitely.

By having oriented channels which can direct blood vessels growth, the channels in the scaffolds structure decrease the distance blood vessels travel through otherwise random angiogenesis to reach the center of the scaffold. They also decrease the amount of blood vessel growth required to vascularize the outer regions of the scaffold, thus rapidly vascularizing a large proportion of the volume of the implant (since x mm of blood vessel growth toward the surface fills a greater volume of scaffold than it would nearer the center).

The phase of cell proliferation begins early on at around 24-48 hours, peaking at around 2-3 weeks. Tissue remodeling begins from around 1-2 weeks. Near complete degradation of the scaffold and tissue regeneration is achieved within 4 weeks.

### Example 2

### Freeze-Sublimation Methods for Constructing Gradient Scaffolding with Varied Pore Diameter

### Preparation of Slurry

Extracellular matrix components, such as, for example, microfibriallar, type I collagen, isolated from bovine tendon (Integra LifeSciences) and chondroitin 6-sulfate, isolated from shark cartilage (Sigma-Aldrich), 10% (w/w) at 1:1 ratio are combined with 0.05M acetic acid at a pH ˜3.2 are mixed at 15,000 rpm, at 4° C., then degassed under vacuum at 50 mTorr.

### Varying Pore Diameter

The suspension is placed in a container, and only part of the container (up to 10% of the length) is submerged in a supercooled silicone bath. The equilibration time for freezing of the slurry is determined, and the freezing process is stopped prior to achieving thermal equilibrium. The container is then removed from the bath and the slurry is then sublimated via freeze-drying (for example, VirTis Genesis freeze-dryer, Gardiner, N.Y.). Thus, a thermal gradient occurs in the slurry, creating a freezing front, which is stopped prior to thermal equilibrium, at which point freeze-drying is conducted, causing sublimation, resulting in a matrix copolymer with a graded average pore diameter field.

In another method, the suspension is placed in a container, on a freezer shelf, where a graded thermal insulation layer is placed between the container and the shelf, which also results in the production of a gradient freezing front, as described above. The graded thermal insulation layer can be constructed by any number of means, including use of materials with varying thermal conductivity, such as aluminum and copper, or aluminum and plexiglass, and others.

In one embodiment, the container is a multicomponent mold, containing removable elements. In one embodiment, removal of these elements following solidification of the polymeric suspension creates tunnels within the frozen slurry, thereby optimizing their orientation. In another embodiment, the removable elements have conical shape, such that in one embodiment, the tunnel diameter narrows the further the distance is from the periphery of the scaffold.

In one embodiment, the surface of the mold creates indentations and channels in the frozen slurry, thereby creating surface folds of desired geometry and distribution across

The foregoing has been a description of certain non-limiting preferred embodiments of the invention. Those of ordinary skill in the art will appreciate that various changes and modifications to this description may be made without departing from the spirit or scope of the present invention, as defined in the following claims.

1. A solid, porous scaffold for implantation, comprising an organic polymer, having a width of at least 3.5 mm in at least one direction, and pores oriented perpendicular to an edge of said scaffold.

2. The scaffold of claim 1, wherein said pores vary in terms of their depth and diameter, which may range from 1-1000 μm respectively.

3. The scaffold of claim 1, wherein said pores are elongated, thereby forming a channel within said scaffold.

4. The scaffold of claim 1, wherein pores situated closer to a surface of said scaffold have a diameter which is greater than pores situated further from said surface.

5. The scaffold of claim 1, wherein said scaffold varies in its average pore diameter, pore size distribution, cross-link density.

6. The scaffold of claim 1, wherein said organic polymer comprises an extracellular matrix protein or an analogue thereof.

7. The scaffold of claim 6, wherein said extracellular matrix protein comprises a collagen, a glycosaminoglycan, or a combination thereof.

8. The scaffold of claim 1, wherein said scaffold further comprises cells, growth factors, cytokines, hormones, inflammatory stimuli, angiogenic factors, or a combination thereof.

9. The scaffold of claim 1, wherein the size and shape of said scaffold is a function of the tissue into which the scaffold is to be implanted.

10. The scaffold of claim 1, wherein said scaffold has a width of between 5-10 mm, in at least one direction.

11. The scaffold of claim 1, wherein said scaffold, when implanted, promotes angiogenesis within, or proximal to said scaffold.

12. The scaffold of claim 1, wherein said scaffold is comprised of a material whose stiffness is sufficient to resist compressive forces of tissue proximal to a site of implantation.

13. A process for preparing a solid, porous, biocompatible scaffold having a width of at least 3.5 mm in at least one direction, and pores oriented perpendicular to an edge of said scaffold, the process comprising the steps of:
a. applying a polymeric suspension to a mold comprised of a conductive material, wherein said mold has at least 2 components;
b. super-cooling the suspension-filled mold in (a) in a refrigerant held at a constant temperature, for a period of time until said suspension is solidified, whereby ice crystals are formed in said solidified polymeric suspension, said crystals being oriented perpendicular to an edge of said scaffold;
c. exposing a portion of said solidified polymeric suspension by removing at least on component of said mold to conditions which enable sublimation in said portion, whereby pores are formed which are perpendicular to an edge of said scaffold; and
d. removing the remaining components of said mold to expose said solid porous scaffold.

14. The process of claim 13, wherein exposing said portion results in said pores being formed within said scaffold are of a non-uniform average diameter.

15. The process of claim 13, wherein the average diameter of said pores formed ranges from 0.1-500 μm.

16. The process of claim 13, wherein said pores formed are elongated, forming a channel within said scaffold.

17. The process of claim 13, wherein said pores formed vary in their distribution in said scaffold.

18. The process of claim 13, further comprising the step of exposing said scaffold to a cross-linking agent.

19. The process of claim 18, wherein said wherein said cross-linking agent is glutaraldehyde, formaldehyde, paraformaldehyde, formalin, (1 ethyl 3-(3-dimethyl aminopropyl)carbodiimide (EDAC), or UV light, or a combination thereof.

20. The process of claim 18, wherein said scaffold varies in terms of its cross-link density.

21. The process of claim 13, wherein said polymeric suspension comprises at least one organic polymer.

22. The process of claim 21, wherein said organic polymer comprises an extracellular matrix protein or an analogue thereof.

23. The process of claim 22, wherein said extracellular matrix protein comprises a collagen, a glycosaminoglycan, or a combination thereof.

24. The process of claim 13, further comprising the step of applying cells, growth factors, cytokines, hormones, inflammatory stimuli, angiogenic factors, or a combination thereof to said scaffold.

25. The process of claim 13, wherein said mold is of a size and shape approximating the tissue into which said scaffold is to be implanted.

26. The process of claim 13, wherein said mold is comprised of two or more conductive materials.

27. The process of claim 26, whereby said conductive materials differ in terms of their local rates of freezing during step (b).

28. The process of claim 13, wherein each component of said mold is comprised of a different conductive material.

29. The process of claim 13, wherein step (b) results in the periphery of said mold being exposed to a common temperature gradient.

30. The process of claim 29, wherein said temperature gradient induces ice crystal nucleation and growth in a direction perpendicular to said mold periphery.

31. The process of claim 30, wherein columnar ice crystals are formed in said scaffold.

32. The process of claim 13, wherein said conductive material, said temperature, immersion time or a combination thereof are varied, to produce varied pore characteristics in said scaffold.

33. The process of claim 13, wherein said process produces a scaffold whose stiffness is sufficient to resist compressive forces of tissue proximal to a site of implantation.

34. A scaffold produced according to the process of claim 13.

35. A method of organ or tissue engineering in a subject, comprising the step of implanting the scaffold of claim 1 in said subject.

36. The method of claim 35, further comprising the step of applying cells to said scaffold.

37. The method of claim 36, wherein said scaffold is cultured for a period of time, prior to implantation into a subject.

38. The method of claim 37, wherein said cells are applied to the periphery of said scaffold.

39. The method of claim 36, wherein said cells are stem or progenitor cells.

40. The method of claim 36, wherein said cells are engineered to express a growth factor, cytokine, hormone, inflammatory stimuli, angiogenic factor, or a combination thereof.

41. The method of claim 36, said engineering is of an organ or tissue comprised of heterogeneous cell types.

42. The method of claim 36, wherein said method is utilized in wound healing.

43. A method of organ or tissue repair or regeneration in a subject, comprising the step of implanting the scaffold of claim 1 in said subject.

44. The method of claim 43, further comprising the step of applying cells to said scaffold.

45. The method of claim 44, wherein said scaffold is cultured for a period of time prior to implantation of said scaffold.

46. The method of claim 44, wherein said cells are applied to the periphery of said scaffold.

47. The method of claim 44, wherein said cells are stem or progenitor cells.

48. The method of claim 44, wherein said cells are engineered to express a growth factor, cytokine, hormone, inflammatory stimuli, angiogenic factor, or a combination thereof.

49. The method of claim 44, wherein said engineering is of an organ or tissue comprised of heterogeneous cell types.

50. The method of claim 44, wherein said method is utilized in wound healing.
